(19) United States
(12) Patent Application Publication
(10) Pub. No.: US 2006/0020254 A1
(43) Pub. Date: Jan. 26, 2006

(54) Suction assisted tissue plication device and method of use

(76) Inventor: Gerard von Hoffmann, Trabuco Canyon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/125,820

(22) Filed: May 10, 2005

Related U.S. Application Data
(60) Provisional application No. 60/569,727, filed on May 10, 2004.

(51) Int. Cl. A61M 31/00

(52) U.S. Cl. 604/500

(57) ABSTRACT

Disclosed are methods and devices for attaching an implant such as a gastrointestinal bypass tube to a position within a tubular organ. Tissue adjacent an attachment site is stabilized using vacuum, and tissue anchors are advanced through the stabilized tissue.

### CROSS-REFERENCE TO RELATED APPLICATIONS

This application claims priority under 35 U.S.C. § 119(e) from United States Provisional Application Ser. No. 60/569,727 filed May 10, 2004, the entirety of which is hereby incorporated by reference.

### BACKGROUND OF THE INVENTION

1. Field of the Invention

The present invention relates generally to surgical devices and methods. More particularly it relates to a device for attaching a tissue attachment structure such as for anchoring a device or for forming and fastening a plication or fold of tissue. The tissue plication can be used to form a stoma or restriction in an organ, such as the stomach, and/or it can be used for attachment of an implant device, such as a gastrointestinal sleeve device for treating morbid obesity and other disorders.

2. Description of the Related Art

The subject matter of this patent application is related to the following commonly owned and copending patent applications, which are hereby incorporated by reference in their entirety, U.S. utility patent application Ser. No. 10/698,148 filed on Oct. 31, 2003 by Kagan et al. for Apparatus and Methods for Treatment of Morbid Obesity and U.S. provisional patent application 60/534,056, filed on Dec. 31, 2003, by Kagan et al. for Devices and Methods for Treating Morbid Obesity. The devices and methods described herein can be used in conjunction with the apparatus and methods described in these prior applications.

### SUMMARY OF THE INVENTION

There is provided in accordance with one aspect of the present invention, an implantable gastrointestinal bypass tube. The tube comprises an elongate flexible tubular body, having a proximal end and a distal end. At least one tissue attachment device is movably carried by the proximal end. At least on suction port is provided, for stabilizing tissue in the vicinity of the tissue attachment device.

The tissue attachment device may be a pin, a t-tag, or other tissue attachment structure. The tissue attachment device may be axially moveably carried along a longitudinal axis which is parallel to the longitudinal axis of the tubular body. Alternatively, the tissue attachment device may be moveable along an axis which is inclined with respect to or transverse to the longitudinal axis of the tubular body, such as for placing transmural attachment devices through the wall of the stomach or esophagus.

In accordance with another aspect of the present invention, there is provided a method of securing a gastrointestinal bypass tube or other gastrointestinal implant to the wall of the stomach. The method comprises the steps of providing an implant, such as an elongate flexible gastrointestinal bypass tube. The implant is positioned with a proximal end in the vicinity of the gastroesophageal junction. Suction is applied to a deployment catheter, to manipulate tissue in the vicinity of a tissue attachment site, and an anchor is deployed into the tissue, to anchor the implant at the sight.

Further features and advantages of the present invention will become apparent to those of ordinary skill in the art in view of the detailed description of preferred embodiments which follows when considered together with the attached drawings and claims.

### BRIEF DESCRIPTION OF THE DRAWINGS

FIG. 1 is a partial cross-sectional fragmentary view of a distal end of a deployment catheter in accordance with the present invention.

FIG. 2 is a longitudinal cross-section through a tubular organ, showing an annular support cuff implanted using the device of FIG. 1.

FIG. 3 is a cross-sectional view through a gastrointestinal bypass tube having an integral vacuum cavity and tissue anchor.

FIG. 4 is a schematic cross-sectional view of an implantable device positioned within a tubular organ.

FIG. 5 is a schematic perspective view of a proximal end of a gastrointestinal bypass tube having tissue anchor apertures.

FIG. 6 is a fragmentary perspective view of a deployment catheter having proximal and distal occlusion balloons, and showing a vacuum port and an anchor deployment port.

### DETAILED DESCRIPTION OF THE PREFERRED EMBODIMENT

A concept that is quite useful in gastrointestinal surgery, as well as other surgical specialties, is the formation of a plication or fold of tissue, such as the wall of an organ, and attaching the plication with a fastener. Optionally, depending on the clinical application, an implant device may be fastened to the tissue plication. The implant device may be fastened to the tissue simultaneously with the fastening of the plication, or it may be fastened to the tissue before or after fastening the plication.

The present invention relates to a surgical device that utilizes suction to assist in creating a full thickness plication of tissue and then attaching the plication with a fastener. In a preferred embodiment, the surgical device is configured as an endoscopic tool with multiple suction chambers or a single annular suction chamber around the periphery of the tool. The tool would also include a fastener delivery and deployment device or multiple devices capable of deploying multiple fasteners in a circular pattern. Suction would be used to draw the tissue into the chamber or chambers to create a plication around the inside of an organ, such as the gastric and/or esophageal wall in the region of the GEJ. Then, the fasteners would be deployed to attach to the plicated tissue. The fasteners may be deployed simultaneously or sequentially. Preferably, the tool is configured to form and fasten a plication around the entire inner periphery of the organ without repositioning the tool. Alternatively, the tool can be configured to form and fasten a plication along a sector of the inner periphery of the organ, requiring the tool to be rotated and actuated one or more times to completely form and fasten a plication around the entire inner periphery of the organ.

The fasteners applied by the tool can include sutures, staples, T-tag fasteners, rivets or other fasteners described herein and in the prior applications noted above. The tool can use buttresses on either side of the plication in conjunction with the fasteners. The fasteners can also be used in conjunction with extragastric buttressing as described in the prior application. The tool can be configured to use a combination of suction plication, extragastric buttress placement and fastener pinning and attachment in a single device.

The tool can be used to create a plication that is used for subsequent attachment of another device, such as a stoma device, mounting ring or gastrointestinal sleeve device. The fasteners used may include hooks, loops or other features to attach or hang another device onto. Alternatively, the tool can be used to attach another device directly to the tissue at the same time as forming and fastening the plication.

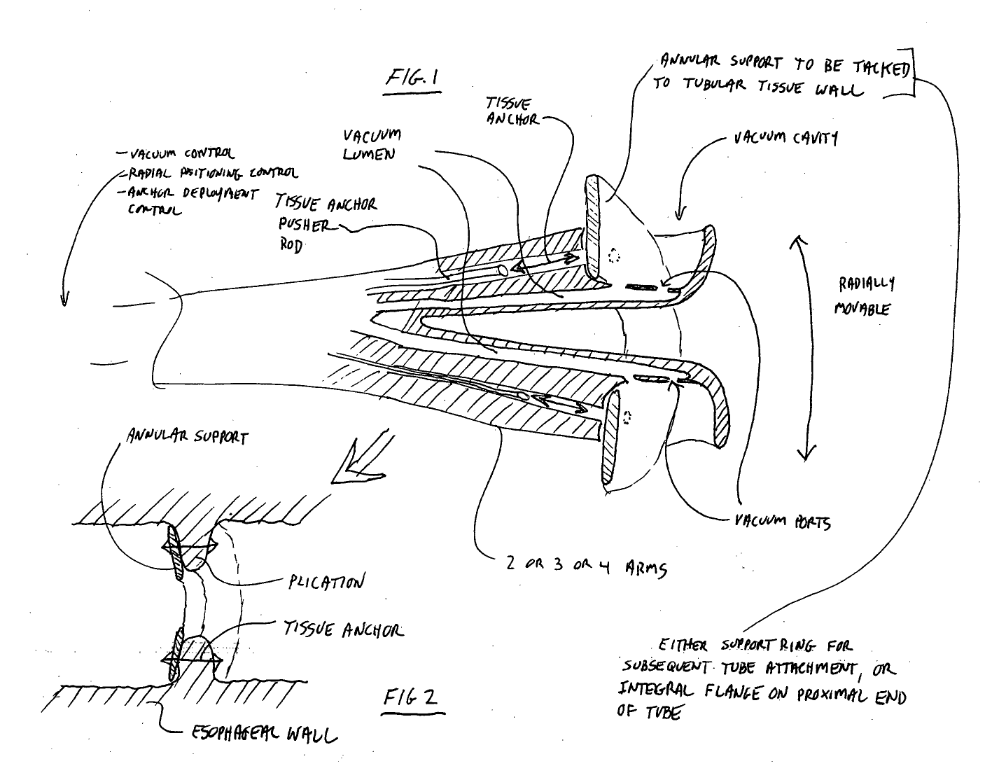

The surgical device can be configured in a number of different ways. In a first embodiment, shown in FIG. 1, a plurality of plication cavities are carried on the distal end of a deployment catheter. They may each be carried on a lever arm with an axially movable control wire so they can be advanced radially outwardly toward the surrounding tissue. Each cavity has one or more vacuum ports which communicate back to a vacuum source. When tissue is drawn into the cavities under vacuum to form a plication, tissue anchors are pushed through the plication and, optionally, through an attachment base or other structure to the tissue wall. Then, the catheter is withdrawn. There is a lot of structural design flexibility on ways to carry this out. FIG. 2 shows a plication formed in the wall of an organ with an annular support to maintain the tissue in the plicated configuration and/or for subsequent attachment of an implant device.

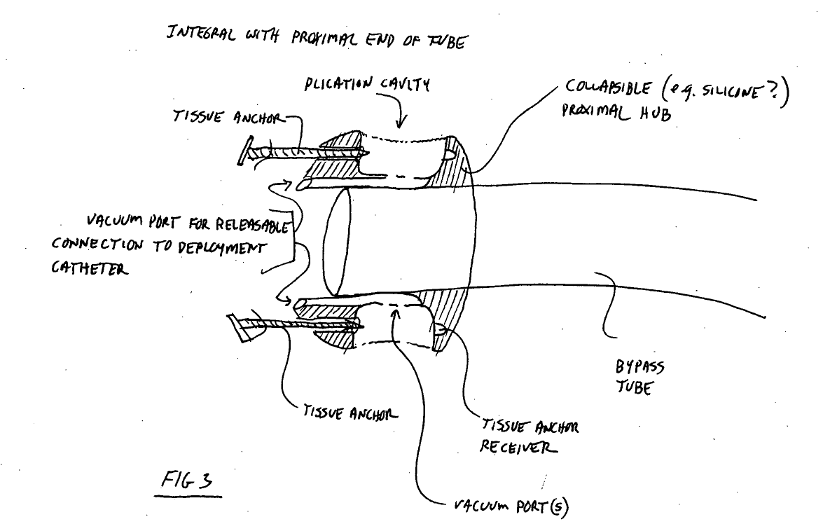

Another approach is to include the plication cavities directly on the implant, as shown in FIG. 3. The implant would also carry one or more tissue darts, which can be advanced across the cavity, for example by pushing a deployment wire on the deployment catheter, which is coaxially aligned with each dart. The vacuum ports are in communication with a vacuum lumen on the deployment catheter across a releasable connection.

Either design could be provided with a pull ring or other structure that could be used later to retract the anchors from the plication and release the implant. This concept would be easily implemented with the second design because those anchors don't need barbs.

If the plication cavities are on the implant like on the second design, then the tissue anchors can be a simple push wire because each end is entrapped in a cavity on the implant. In the first design, the axial holding power of the tissue anchor may be a limiting feature especially in really soft tissue.

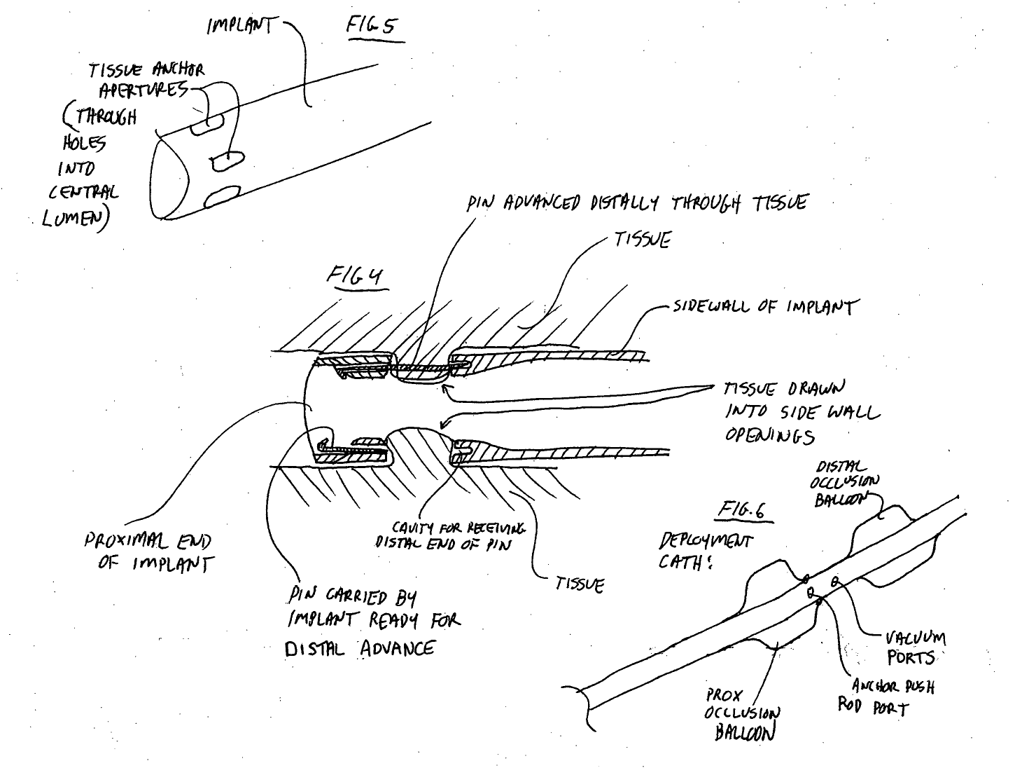

FIGS. 4-5 show another embodiment of the present invention. The proximal end of an implant, such as a gastrointestinal sleeve device for treating morbid obesity, is configured with anchoring apertures through the sidewall. This is one of the lowest profile designs. The deployment catheter, shown in FIG. 6, has two occlusion balloons which are positioned proximally and distally of the anchoring apertures and inflated. The occlusion balloons are preferably made of a highly elastic material, such as silicone, to create a good seal without too much dilatation. Suction can be applied through a lumen to a suction port on the catheter sidewall in between the two balloons, to draw tissue through the anchoring apertures. This would draw tissue into all of the anchoring apertures simultaneously. Then, one or more push rods on the catheter are advanced distally to advance a pin across each anchoring aperture. The catheter is then deflated and withdrawn. FIG. 4 shows the device being attached within an organ (the deployment catheter has been omitted from the drawing for clarity). At the bottom of FIG. 4, the vacuum has been applied to draw tissue in through the aperture and the pin is ready for distal advancement. At the top of FIG. 4, the pin has been advanced distally to fasten the device to the tissue.

Direct visualization may be desirable, using endoscopy, fluoroscopy and/or ultrasound, to confirm that sufficient tissue has been drawn in, prior to advancing the push rod. The proximal ends of the pins could be connected together by a ring that is axially movable to advance or retract the pins as a single unit. That would make it easier to remove the device, by hooking the ring with a removal catheter and pulling it proximally to simultaneously retract the tissue pins. The device can use as many apertures and pins as necessary for the clinical application, based upon experimentation. By way of example, the device can use 4 apertures spaced 90 degrees apart around the periphery of the device. There could be two or more rings of apertures in more than one transverse plane, if needed for adequate support. The tissue drawn through the apertures would thereby be exposed to the interior of the device following implantation. This exposure along with the dimensions of the aperture may help to avoid tissue necrosis. The pins could be curved and concave in a radially inward direction, to allow them to track a curved path out a little bit, to grab even deeper into the adjacent tissue wall. Axially extending slits could be provided from the proximal end of the device extending distally in between circumferentially adjacent apertures, to provide lever arms or hinged portions, so the tissue anchors could flex radially in and out in response to diameter changes in the intestine or stomach. This could also allow the operator to reduce the crossing profile during deployment, and/or overextend the device radially during attachment to reach out and grab the adjacent tissue. The whole proximal end can be resilient and crushed or folded onto the deployment catheter and restrained by an outer tubular sheath.

The devices and methods described herein can also be configured to form a plurality of plications simultaneously. For example the surgical device can be configured to form a double plication in the tissue wall with an implant device attached between the two plications.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. An implantable gastrointestinal bypass tube, comprising:
an elongate, flexible tubular body, having a proximal end and a distal end;
at least one tissue attachment device, movably carried by the proximal end; and
at least one suction port, for stabilizing tissue in the vicinity of the tissue attachment device.
